(19) United States
(12) Patent Application Publication
(10) Pub. No.: US 2025/0172548 A1
(43) Pub. Date: May 29, 2025

(54) METHODS AND SYSTEMS FOR USING ISOTOPICALLY LABELED ANALYTES FOR CALIBRATION FOR QUANTIFICATION OF ANALYTES IN DRIED SAMPLES

(71) Applicant: LABORATORY CORPORATION OF AMERICA HOLDINGS, Burlington, NC (US)

(72) Inventor: Russell Phillip Grant, Chapel Hill, NC (US)

(21) Appl. No.: 18/950,857

(22) Filed: Nov. 18, 2024

Related U.S. Application Data
(60) Provisional application No. 63/599,953, filed on Nov. 16, 2023.

(51) Int. Cl. G01N 33/543; G01N 1/40; G01N 27/623

(52) U.S. Cl. CPC G01N 33/543; G01N 1/40; G01N 27/623; G01N 2458/15

(57) ABSTRACT

Disclosed are methods and systems to measure the amount of an analyte in a dried sample. The method includes adding a known amount of an isotopically labeled version of the analyte of interest to a solid substrate, wherein the isotopically labeled version of the analyte of interest is the analyte of interest labeled with at least one stable isotope. The method may further include adding the biological sample to the substrate and allowing the biological sample to dry on the substrate. The method may also include measuring an amount of the analyte of interest and the isotopically labeled version of the analyte of interest present on the substrate and determining the amount of the analyte of interest present in the biological sample based on the amounts of the analyte of interest and the isotopically labeled version of the analyte of interest.

### FIELD OF THE INVENTION

The present disclosure generally relates to methods and systems for using isotopically labeled analytes for calibration when quantifying analytes in a sample. More specifically, the present disclosure relates to methods and systems for using one or more isotopically labeled analytes for calibration when quantifying analytes in dried samples using mass spectrometry.

### BACKGROUND

Currently, there are platforms available for the procurement of biological samples (e.g., blood) that can be self-collected by an individual. These platforms, such as microsampling devices, enable quantitative collection of a liquid biological sample that is then allowed to dry on a solid substrate, such that the dried sample may be sent directly to a clinical laboratory for measurement of biomarkers of interest. The ability of an individual to collect his or her own biological sample (e.g., blood or urine) on a solid substrate has the potential to revolutionize the healthcare industry. Rather than going to a doctor's office to provide a biological sample, the individual may simply self-collect the biological sample and mail it to a lab for testing. This can be very beneficial, for example, for routine follow-up testing or monitoring of a medical condition.

There are, however, challenges associated with clinical measurement of analytes from dried samples. For example, clinical measurement of a biomarker requires accurately detecting the presence of the biomarker over a range of potential concentrations (i.e., lack of false negatives and/or false positives), and also requires detecting the biomarker with quantitative accuracy (i.e., determination of the amount of a biomarker). For example, for testosterone, the acceptable values for desirable total error, imprecision, and bias using intra and inter-individual variance specifications is 13.61%, 4.63%, and 5.98, respectively.

Although self-collection devices are convenient and allow for reproducible sample collection, there can be challenges to utilize dried samples for quantitative and precise laboratory analysis so as to provide a clinically relevant assessment from the sample. For example, extraction of an analyte from a dried sample may not be quantitative, requiring the need for complex internal calibration procedures. Prior methods of using a normalizing analyte that is present in a sample at a known concentration but that is distinct from the analyte (e.g., chloride ion in blood) has been described (e.g., U.S. Patent Publication No. 2019/0369113). However, selection and measurement of an appropriate normalizing agent for individual analytes can be technically challenging. Thus, there is a need for simplified, high throughput assays for clinical testing. Such assays should allow for ease of sample procurement, but still provide accuracy in testing.

### SUMMARY

Disclosed are methods and systems to measure the amount of an analyte of interest in a dried sample. The disclosed methods and systems may be embodied in a variety of ways.

In some embodiments, the present disclosure provides a method to measure the amount of an analyte of interest present in a dried biological sample comprising: adding a known amount of a first isotopically labeled version of the analyte of interest to a solid substrate, wherein the isotopically labeled version of the analyte of interest is the analyte of interest labeled with at least one stable isotope. The method may also include the steps of adding the biological sample to the solid substrate and allowing the biological sample to dry on the solid substrate. Additionally, the method may include measuring an amount of the analyte of interest and the isotopically labeled version of the analyte of interest present on the solid substrate and determining the amount of the analyte of interest present in the biological sample based on the amounts of the analyte of interest and the isotopically labeled version of the analyte of interest.

In some embodiments, the present disclosure provides systems to measure the amount of an analyte of interest in a dried sample. The system may comprise a device for measuring the amount of an analyte of interest present in a biological sample comprising at least one solid substrate, wherein a known amount of an isotopically labeled version of the analyte of interest is applied to the solid substrate, and wherein the isotopically labeled version of the analyte of interest is the analyte of interest labeled with at least one stable isotope.

### BRIEF DESCRIPTION OF THE DRAWINGS

The invention may be better understood by reference to the following non-limiting figures.

FIG. 1 (Prior Art) shows a graph of a calibration curve generated using external calibration with internal standardization.

FIG. 2 provides a flowchart of a method of measuring an amount of an analyte of interest present in a dried biological sample on a solid substrate in accordance with an embodiment of the disclosure.

FIG. 3A shows a graph of peak responses for an analyte of interest (testosterone) and an isotopically labeled version of the analyte of interest (testosterone-[¹³C₃]) for internal calibration in accordance with embodiments of the disclosure.

FIG. 3B shows a graph of peak responses for an analyte of interest (testosterone) and an isotopically labeled version of the analyte of interest (testosterone-[¹³C₃]) with two separate MS/MS transitions that appear at different peak intensities in accordance with embodiments of the disclosure.

FIG. 4 shows a graph of multiple responses for internal standards generated using both minor mass offsets for precursor to product ion transitions for an isotopically labeled version of the analyte of interest and varying collision energies to generate multiple responses for a single internal standard that correspond to different equivalent analyte concentrations in accordance with an embodiment of the disclosure.

FIG. 5 shows a graph of isotopically labeled internal calibrator (ILIC) responses that are individually calibrated against the analyte concentrations in Table 1 to generate an internal calibrator concentration for each response that spans a concentration range in accordance with an embodiment of the disclosure.

FIG. 6 shows a graph of responses for isotopically labeled internal calibrators (ILIC) of isotopically labeled version of testosterone (testosterone internal standard (IS)) measured under conditions to generate different equivalent analyte concentrations as shown in FIG. 5, where the equivalent analyte concentrations are selected for relevance to medical decision points for testosterone levels in male (M) and female (F) (both pre- and post-menopausal) subjects in accordance with an embodiment of the disclosure.

FIG. 7 shows a system in accordance with an embodiment of the disclosure.

FIG. 8A shows a scatter plot obtained using the lowest response of internal standard (isotopically labelled internal calibrator (ILIC)), at 4.54 ng/dL, (y-axis) measured in FIG. 5 versus the CDC reference method samples (ng/dL) (x-axis).

FIG. 8B shows a Bland Altman plot of the percent bias using the lowest response of internal standard (ILIC) at 4.54 ng/dL measured in FIG. 5 to quantify the CDC reference method samples.

FIG. 9A shows a scatter plot obtained using the highest response of internal standard (ILIC) at 3911 ng/dL (y-axis) measured in FIG. 5 versus the CDC reference method samples (ng/dL) (x-axis).

FIG. 9B shows a Bland Altman plot of the percent bias using the highest response of internal standard (isotopically labelled internal calibrator) at 3911 ng/dL measured in FIG. 5 to quantify the CDC reference method samples.

FIG. 10A shows a scatter plot obtained using the mean response of the analyte ratio for all six (6) internal standards (ILICs, ng/dL) (y-axis) measured in FIG. 5 versus the CDC reference method samples (ng/dL) (x-axis).

FIG. 10B shows a Bland Altman plot of the percent bias using the mean response of the six (6) internal standards (ILICs) measured in FIG. 5 to quantify the CDC reference method samples (ng/dL).

FIG. 11A shows a graph comparing isotope dilution and a reference method to calculate the concentration of an analyte using internal standards in accordance with an embodiment of the disclosure.

FIG. 11B shows a reference method to calculate the concentration of an analyte using internal standards in accordance with an embodiment of the disclosure.

### DETAILED DESCRIPTION

The present disclosure now will be described more fully hereinafter. The disclosure may be embodied in many different forms and should not be construed as limited to the aspects set forth herein: rather, these aspects are provided so that this disclosure will satisfy applicable legal requirements. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section or as used elsewhere herein prevails over the definition that is incorporated herein by reference.

Many modifications and other embodiments of the disclosed subject matter set forth herein will come to mind to one skilled in the art to which the disclosed subject matter pertains having the benefit of the teachings presented in the description. Therefore, it is to be understood that the disclosed subject matter is not to be limited to the specific embodiments disclosed herein and that modifications and other embodiments are intended to be included within the scope of the appended claims. Although specific terms are employed herein, they are used in a generic and descriptive sense only and not for purposes of limitation.

### Definitions

While the following terms are believed to be well understood by one of ordinary skill in the art, the following definitions are set forth to facilitate explanation of the presently disclosed subject matter. Other definitions are found throughout the specification. Unless otherwise defined, all technical and scientific terms used herein have the same meaning as commonly understood by one of ordinary skill in the art to which this presently described subject matter belongs.

When introducing elements of the present disclosure or the embodiment(s) thereof, the articles “a,” “an,” “the” and “said” are intended to mean that there are one or more of the elements. The terms “comprising,” “including” and “having” are intended to be inclusive and mean that there may be additional elements other than the listed elements. It is understood that aspects and embodiments of the disclosure described herein include “consisting” and/or “consisting essentially of” aspects and embodiments.

The term “and/or” when used in a list of two or more items, means that any one of the listed items can be employed by itself or in combination with any one or more of the listed items. For example, the expression “A and/or B” is intended to mean either or both of A and B. i.e., A alone, B alone or A and B in combination. The expression “A, B and/or C” is intended to mean A alone, B alone, C alone, A and B in combination, A and C in combination, B and C in combination or A, B, and C in combination.

The term “about” is used to indicate that a value includes the inherent variation of error for the device, the method being employed to determine the value, or the variation that exists among sample.

Various aspects of this disclosure are presented in a range format. It should be understood that the description in range format is merely for convenience and brevity and should not be construed as an inflexible limitation on the scope of the disclosure. Accordingly; the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range.

As used herein, an analyte is a molecule or biological compound being analyzed either qualitatively (e.g., for identification) or quantitatively (e.g., to determine a relative or absolute amount).

As used herein, a “subject” or “individual” are used interchangeably and may comprise an animal. Thus, in some embodiments, a sample obtained from a subject is obtained from a mammalian animal, including, but not limited to a human or fetus, a dog, a cat, a horse, a rat, a monkey, and the like. In some embodiments, the sample or biological sample is obtained from a human subject. In some embodiments, the subject is a patient, that is, a living person presenting themselves in a clinical setting for diagnosis, prognosis, or treatment of a disease or condition.

“Sample” or “patient sample” or “biological sample” or “specimen” are used interchangeably herein. Non-limiting examples of samples that may be dried for analysis with the disclosed systems and methods include blood or a blood product (e.g., serum, plasma, or the like), urine, nasal swabs, saliva, a liquid biopsy sample (e.g., for the detection of cancer), or combinations thereof. The term “blood” encompasses whole blood, a blood product or any fraction of blood, such as serum, plasma, buffy coat, or the like as conventionally defined. Suitable samples include those which are capable of being deposited onto a substrate for collection and drying including, but not limited to: blood, plasma, serum, urine, saliva, tear, cerebrospinal fluid, organ, hair, muscle, or other tissue sample or other liquid aspirate. In an embodiment, the sample body fluid may be separated on the substrate prior to drying. For example, blood may be deposited onto a sampling paper substrate which limits migration of red blood cells allowing for separation of the blood plasma fraction prior to drying in order to produce a dried plasma sample for analysis.

As used herein, the term “chromatography” refers to a process in which a chemical mixture carried by a liquid or gas is separated into components as a result of differential distribution of the chemical entities as they flow around or over a stationary liquid or solid phase.

As used herein, the phrase “liquid chromatography” or “LC” is used to refer to a process for the separation of one or more molecules or analytes in a sample from other analytes in the sample. LC involves the slowing of one or more analytes of a fluid solution as the fluid uniformly moves through a column of a finely divided substance. The slowing results from the distribution of the components of the mixture between one or more stationary phases and the mobile phase. LC includes, for example, reverse phase liquid chromatography (RPLC) and high performance liquid chromatography (HPLC). In some cases. LC refers to reverse phase LC with a hydrophobic stationary phase in combination with a mobile phase comprised of water and/or water-miscible organic solvents, such as methanol or acetonitrile. In some case. LC may refer to ion exchange chromatography, affinity chromatography, normal phase liquid chromatography, or hydrophilic interaction chromatography.

As used herein, the term “HPLC” or “high performance liquid chromatography” refers to liquid chromatography in which the degree of separation is increased by forcing the mobile phase under pressure through a stationary phase, typically a densely packed column. The chromatographic column typically includes a medium (i.e., a packing material) to facilitate separation of chemical moieties (i.e., fractionation). The medium may include minute particles. The particles can include a bonded surface that interacts with the various chemical moieties to facilitate separation of the chemical moieties such as the biomarker analytes quantified in the experiments herein. One suitable bonded surface is a hydrophobic bonded surface such as an alkyl bonded surface. Alkyl bonded surfaces may include C-4. C-8, or C-18 bonded alkyl groups, preferably C-18 bonded groups. The chromatographic column includes an inlet port for receiving a sample and an outlet port for discharging an effluent that includes the fractionated sample. In the method, the sample (or pre-purified sample) may be applied to the column at the inlet port, eluted with a solvent or solvent mixture, and discharged at the outlet port. Different solvent modes may be selected for eluting different analytes of interest. For example, liquid chromatography may be performed using a gradient mode, an isocratic mode, or a polytyptic (i.e. mixed) mode.

As used herein, the term “HTLC” refers to high turbulence liquid chromatography Liquid chromatography may, in certain embodiments, comprise high turbulence liquid chromatography or high throughput liquid chromatography (HTLC). See, e.g., Zimmer et al., J. Chromatogr. A 854:23-35 (1999); see also, U.S. Pat. Nos. 5,968,367; 5, 919,368; 5,795,469; and 5,772,874. Traditional HPLC analysis relies on column packings in which laminar flow of the sample through the column is the basis for separation of the analyte of interest from the sample. In such columns, separation is a diffusional process. Turbulent flow, such as that provided by HTLC columns and methods, may enhance the rate of mass transfer, improving the separation characteristics provided.

As used herein, the terms “isolate.” “separate.” or “purify” or the like are not used necessarily to refer to the removal of all materials other than the analyte of interest from a sample matrix. Instead, in some embodiments, the terms are used to refer to a procedure that enriches the amount of one or more analytes of interest relative to one or more other components present in the sample matrix. In some embodiments, a “separation” or “purification” may be used to remove or decrease the amount of one or more components from a sample that could interfere with the detection of the analyte, for example, by mass spectrometry.

As used herein, the term “mass spectrometry” or “MS” refers to a technique for the identification and/or quantitation of molecules in a sample. MS includes ionizing the molecules in a sample to form charged molecules (ions) in gas phase; separating the charged molecules according to their mass-to-charge (m/z) ratio; and detecting the charged molecules. MS allows for both the qualitative and quantitative detection of molecules in a sample. The molecules may be ionized and detected by any suitable means known to one of skill in the art. As used herein, a “mass spectrometer” is an apparatus that includes a means for ionizing molecules and detecting charged molecules.

In certain embodiments. “tandem mass spectrometry” (MS/MS) is used. Tandem mass spectrometry (MS/MS) is the name given to a group of mass spectrometric methods wherein “parent or precursor” ions generated from a sample are fragmented to yield one or more “fragment, daughter or product” ions, which are subsequently mass analyzed by a second MS procedure. As used herein, parent and precursor ion are used interchangeably. Also as used herein, fragment, daughter and product ions are used interchangeably. MS/MS methods are useful for the analysis of complex mixtures, especially biological samples, in part because the selectivity of MS/MS can minimize the need for extensive sample clean-up prior to analysis. In an example of an MS/MS method (i.e., triple quadrupole MS/MS), precursor ions are generated from a sample and passed through a first mass filter (quadrupole 1 or Q1) to select those ions having a particular mass-to-charge ratio. These ions are then fragmented, typically by collisions with neutral gas molecules in the second quadrupole (Q2), to yield product (fragment) ions which are selected in the third quadrupole (Q3), the mass spectrum of which is recorded by an electron multiplier detector. The product ion spectra so produced are indicative of the structure of the precursor ion, and the two stages of mass filtering can eliminate ions from interfering species present in the conventional mass spectrum of a complex mixture.

The term “ionization” and “ionizing” as used herein refers to the process of generating an analyte ion having a net electrical charge equal to one or more electron units. Negative ions are those ions having a net negative charge of one or more electron units, while positive ions are those ions having a net positive charge of one or more electron units.

The term “electron ionization” as used herein refers to methods in which an analyte of interest in a gaseous or vapor phase interacts with a flow of electrons. Impact of the electrons with the analyte produces analyte ions, which may then be subjected to a mass spectrometry technique.

The term “chemical ionization” as used herein refers to methods in which a reagent gas (e.g. ammonia) is subjected to electron impact, and analyte ions are formed by the interaction of reagent gas ions and analyte molecules.

The term “matrix-assisted laser desorption ionization,” or “MALDI” as used herein refers to methods in which a non-volatile sample is exposed to laser irradiation, which desorbs and ionizes analytes in the sample by various ionization pathways, including photo-ionization, protonation, deprotonation, and cluster decay: For MALDI, the sample is mixed with an energy-absorbing matrix, which facilitates desorption of analyte molecules.

The term “surface enhanced laser desorption ionization,” or “SELDI” as used herein refers to another method in which a non-volatile sample is exposed to laser irradiation, which desorbs and ionizes analytes in the sample by various ionization pathways, including photo-ionization, protonation, deprotonation, and cluster decay. For SELDI, the sample is typically bound to a surface that preferentially retains one or more analytes of interest. As in MALDI, this process may also employ an energy-absorbing material to facilitate ionization.

The term “electrospray ionization,” or “ESI,” as used herein refers to methods in which a solution is passed along a short length of capillary tube, to the end of which is applied a high positive or negative electric potential. Upon reaching the end of the tube, the solution may be vaporized (nebulized) into a jet or spray of very small droplets of solution in solvent vapor. This mist of droplet can flow through an evaporation chamber which is heated slightly to prevent condensation and to evaporate solvent. As the droplets get smaller the electrical surface charge density increases until such time that the natural repulsion between like charges causes ions as well as neutral molecules to be released.

The term “Atmospheric Pressure Chemical Ionization,” or “APCI,” as used herein refers to mass spectroscopy methods that are similar to ESI, however, APCI produces ions by ion-molecule reactions that occur within a plasma at atmospheric pressure. The plasma is maintained by an electric discharge between the spray capillary and a counter electrode. Then, ions are typically extracted into a mass analyzer by use of a set of differentially pumped skimmer stages. A counterflow of dry and preheated N₂ gas may be used to improve removal of solvent. The gas-phase ionization in APCI can be more effective than ESI for analyzing less-polar species.

The term “Atmospheric Pressure Photoionization” (“APPI”) as used herein refers to the form of mass spectroscopy where the mechanism for the photoionization of molecule M is photon absorption and electron ejection to form the molecular M+. Because the photon energy typically is just above the ionization potential, the molecular ion is less susceptible to dissociation. In many cases it may be possible to analyze samples without the need for chromatography, thus saving significant time and expense. In the presence of water vapor or protic solvents, the molecular ion can extract H to form MH+. This tends to occur if M has a high proton affinity. This does not affect quantitation accuracy because the sum of M+ and MH+ is constant.

As used herein, the term “isotopically labeled,” “stable isotopically labeled” or “stable isotope labeled” or similar such terms encompasses the process or product, respectively, of enriching a molecule with a non-radioactive isotope of a given atom so as to alter the average mass of said atom within a molecule and thereby alter the average mass of said molecule. Generally, this is accomplished by replacing the light isotopes more frequently found in nature and in natural molecules (e.g., carbon-12 (¹²C) or nitrogen-14 (¹⁴N)), with the less common heavy isotopes (e.g., carbon-13 (¹³C) or nitrogen-15 (¹⁵N)).

As used herein, a “quadrupole analyzer” is a type of mass analyzer used in MS. It consists of four circular rods (two pairs) that are set highly parallel to each other. The quadrupole may be in triple quadrupole format as is known in the art. The quadrupole analyzer is the component of the instrument that organizes the charged particles of the sample based on their mass-to-charge ratio. One of skill in the art would understand that use of a quadrupole analyzer can lead to increased specificity of results. One pair of rods is set at a positive electrical potential and the other set of rods is at a negative potential. To be detected, an ion must pass through the center of a trajectory path bordered and parallel to the aligned rods. When the quadrupoles are operated at a given amplitude of direct current and radio frequency voltages, only ions of a given mass-to-charge ratio will resonate and have a stable trajectory to pass through the quadrupole and be detected. As used herein. “positive ion mode” refers to a mode wherein positively charged ions are detected by the mass analyzer, and “negative ion mode” refers to a mode wherein negatively charged ions are detected by the mass analyzer.

As used herein, selected reaction monitoring (SRM) refers to the technique of using tandem mass spectrometry to select and measure a particular fragment ion of a selected precursor ion. For “selected ion monitoring” or “SIM.” the amplitude of the direct current and the radio frequency voltages are set to observe only a specific mass.

As used herein, the term multiple reaction monitoring (MRM) refers to the technique of using tandem mass spectrometry to select and measure more than one parent/precursor and fragment/product pairs within a given analysis. MRM is the application of SRM to multiple product ions from one or more precursor ions.

### Methods for Quantifying Analytes in a Dried Sample

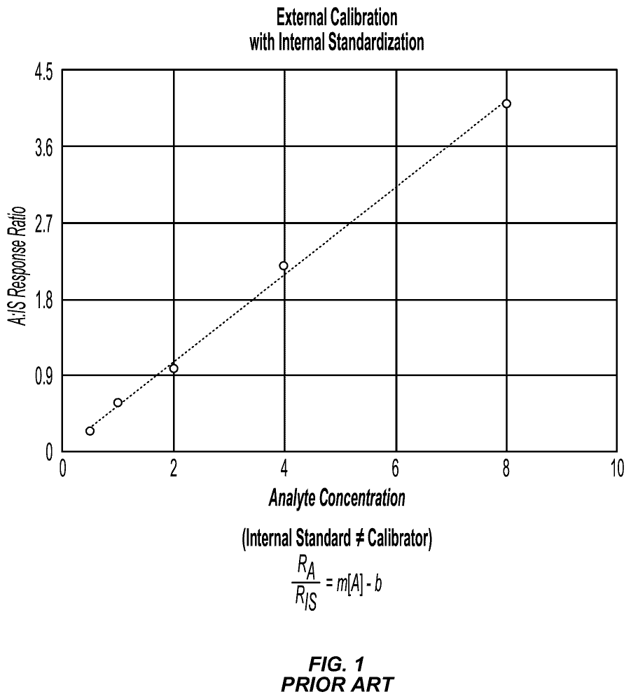

Quantitative mass spectrometry routinely employs external calibration with internal standardization as a calibration scheme for quantitative analysis of unknown analytes in a sample. For example, a series of calibrators are prepared at increasing concentrations of analyte and mixed with an isotopically labeled form of the analyte added at a single concentration. As shown in FIG. 1 (PRIOR ART), a calibration curve plot is created whereby the concentration of analyte in each calibrator is plotted on the x-axis and the ratio of measured analyte peak area or response divided by the measured isotopically labeled internal standard (Ar:IS) is plotted on the y-axis. Notably, the internal standard concentration is not considered for producing the calibration curve and is assigned a value of 1 (i.e., only the analyte concentration itself is listed on the x-axis). The best fit line of the relationship between analyte concentration of the calibrators (x-axis) and the ratio of increasing integrated peak area or response of analyte and isotopically labeled internal standard (y-axis) is used to generate a calibration equation (i.e., R_{A}/R_{IS}=m[A]−b). In this equation, m is the slope, X is the concentration of analyte [A] and b is the y-axis intercept. Unknown samples are extracted with the same amount of isotopically labeled internal standard added and subsequently analyzed by mass spectrometry to determine a ratio of analyte to internal standard peak area or response ratio R_{A}/R_{IS}. The concentration of analyte within the sample can be determined from the calibration curve equation by rearrangement to solve for the concentration of analyte [A]. However, the use of external calibration with internal standardization requires additional calibrators to be prepared and analyzed along with test samples, requiring additional processing time and cost. This step can be a significant cost burden when extracting dried samples from solid substrates.

The present disclosure provides methods for internal calibration to quantify one or more analytes in a dried sample for simplified, high throughput assays for clinical testing. In some embodiments, disclosed are methods to measure an amount of an analyte of interest in a dried sample. For example, a biological sample can be provided to a substrate to produce a dried sample. The substrate can be a solid substrate. In some embodiments, the solid substrate can be a collection card. The collection card can include a matrix to absorb a biological sample. For example, the collection card can receive a blood sample to provide a dried blood spot (e.g., a dried blood spot card). In some embodiments, the dried sample is a dried whole blood sample. In some embodiments, the dried sample is one or more separated components from a whole blood sample (e.g., plasma, serum, red blood cells, white blood cells, and/or platelets). The separated components from the whole blood sample can be provided in different regions of the substrate. The disclosed methods may be embodied in a variety of ways.

In some embodiments, disclosed is a method to measure the amount of an analyte of interest present in a dried biological sample comprising: adding a known amount of a first isotopically labeled analyte of interest to a solid substrate, wherein the isotopically labeled version of the analyte of interest is the analyte of interest labeled with at least one stable isotope: adding the biological sample to the solid substrate: allowing the biological sample to dry on the solid substrate: measuring an amount of the analyte of interest and the isotopically labeled version of the analyte of interest present on the solid substrate; and determining the amount of the analyte of interest present in the biological sample based on the measured amounts of the analyte of interest and the isotopically labeled version of the analyte of interest.

As discussed in more detail herein, the method may comprise adding one or more isotopically labeled forms of the analyte of interest to the solid substrate. For example, one or more isotopically labeled forms of the analyte of interest can be deposited (e.g., pipetted) onto a region of the solid substrate. The region of the solid substrate that includes the one or more isotopically labeled forms of the analyte of interest can be the same region that a biological sample is provided to the solid substrate. In certain embodiments, the method may comprise adding an additional isotopically labeled analyte of interest (e.g., a second isotopically labeled form of the analyte) to the solid substrate. The additional isotopically labeled version of the analyte of interest may comprise the analyte of interest labeled with an additional isotope that is the same as the at least one isotope and/or a different stable isotope or isotopes. In certain embodiments, the additional isotopically labeled version of the analyte of interest is present at a different concentration than the first isotopically labeled version of the analyte of interest. In certain embodiments, multiple isotopes (e.g., 2, 3, 4, 5 or more) may be used for any of the isotopically labeled analytes of interest. The multiple isotopes may be the same isotope (e.g., multiple deuterium or ²D atoms) or different isotopes (e.g., a combination of ²D, ¹³C and/or ¹⁵N).

In some embodiments, a first isotopically labeled version of the analyte of interest and a second isotopically labeled version of the analyte of interest can be provided to a solid substrate. The first isotopically labeled version of the analyte of interest and the second isotopically labeled version of the analyte of interest can be the same isotope of the analyte of interest. Although the first isotopically labeled version of the analyte of interest and the second isotopically labeled version of the analyte of interest are the same isotope of the analyte of interest, each isotope can be provided to the solid substrate in the same or different quantities in different regions of the substrate. For example, if the analyte of interest is testosterone, the first isotopically labeled version of the analyte of interest and the second isotopically labeled version of the analyte of interest can be ¹³C₃-testosterone. The first isotopically labeled version of the analyte of interest and the second isotopically labeled version of the analyte of interest can be added to the solid substrate in different quantities in different regions of the substrate.

In some embodiments, a single isotopically labeled version of the analyte of interest can be provided to a solid substrate. The single isotopically labeled version of the analyte of interest can be deposited on one or more locations of the solid substrate. For example, the isotopically labeled version of the analyte of interest can be ¹³C₃-testosterone. The ¹³C₃-testosterone label can be deposited on a plurality of locations on the solid substrate at the same or different concentrations. In some embodiments, the isotopically labeled version of the analyte of interest can be deposited at different concentrations on the solid substrate. For example, the isotopically labeled version of the analyte of interest can be provided at different concentrations in two or more locations on a card or a dried matrix.

In some embodiments, the single isotopically labeled version of the analyte of interest can be used to generate a plurality of peak areas or responses using a mass spectrometer. For example, single isotopically labeled version of the analyte of interest can be used to generate a plurality of product ions. The ratio of the product ions of the single isotopically labeled version of the analyte of interest can be determined for a known concentration of the isotopically labeled version of the analyte of interest. A precursor ion of an isotopically labeled version of the analyte of interest can be used to generate multiple product ion transitions from the same precursor with different integrated peak areas or responses of different intensities. The product ion transitions can be assigned a concentration at different levels. In some embodiments, the product ion transitions can be assigned a concentration at two discrete levels, for example, above and below the response observed for analyte integrated peak area. The different internal calibrator responses, derived from two different transitions, can be used to calculate the analyte concentration.

In some embodiments, a first isotopically labeled version of the analyte of interest and a second isotopically labeled version of the analyte of interest can be provided to a solid substrate. The first isotopically labeled version of the analyte of interest and the second isotopically labeled version of the analyte of interest can be different isotopes of the analyte of interest. For example, if the analyte of interest is testosterone, the first isotopically labeled version of the analyte of interest can be ¹³C-testosterone, and the second isotopically labeled version of the analyte of interest can be ²D-testosterone. Additionally, and/or alternatively, the method may comprise measuring one or more analytes of interest. For example, the method may comprise adding a known amount of a plurality of isotopically labeled analytes of interest to a solid substrate, wherein the plurality of isotopically labeled analytes of interest each comprises one of the analytes of interest labeled with at least one stable isotope.

In some embodiments, the analyte(s) of interest and the isotopically labeled analyte(s) are extracted from the solid substrate prior to measuring an amount of the analyte(s) of interest and the isotopically labeled analyte(s) of interest present on the substrate. For example, a isotopically labeled version of an analyte of interest can be deposited onto a substrate (e.g., a blood collection card) prior to addition of sample including the analyte of interest. In some embodiments, an isotopically labeled version of an analyte of interest can be provided in an extraction solution to extract the sample from the substrate. In some embodiments, the substrate may include a known amount of an isotopically labeled version of an analyte of interest and the extraction solution may include a known amount of an isotopically labeled version of an analyte of interest.

By predosing the card (or other solid substrate) with the isotopically labeled version of the analyte of interest, the method ensures both the analyte of interest and the isotopically labeled analyte are subjected to the same conditions for subsequent isolation and measurement. In conventional methods, an internal standard (e.g., such as an isotopically labeled version of the analyte of interest) is added to a solution that is used for extraction of the analyte of interest from the solid substrate (e.g., card). However, adding the internal standard to the extraction solution requires that the analyte of interest be quantitatively (i.e., 100% efficiency) extracted from the solid substrate. Therefore, the prior methods of calibration may not provide accurate quantitation of an analyte of interest.

In an embodiment, the biological sample added to the substrate comprises a known volume. In this way, the measured analyte and isotopically labeled analyte(s) may be directly correlated to a known sample volume. For example, using a Capitainer®B microsampling card, a subject may self-collect a blood sample that is captured as a known volume (e.g., 10 μL). Other commercial sampling devices may be used to deposit known volumes of other fluid samples (e.g., blood, plasma, serum, saliva or urine) on a solid substrate.

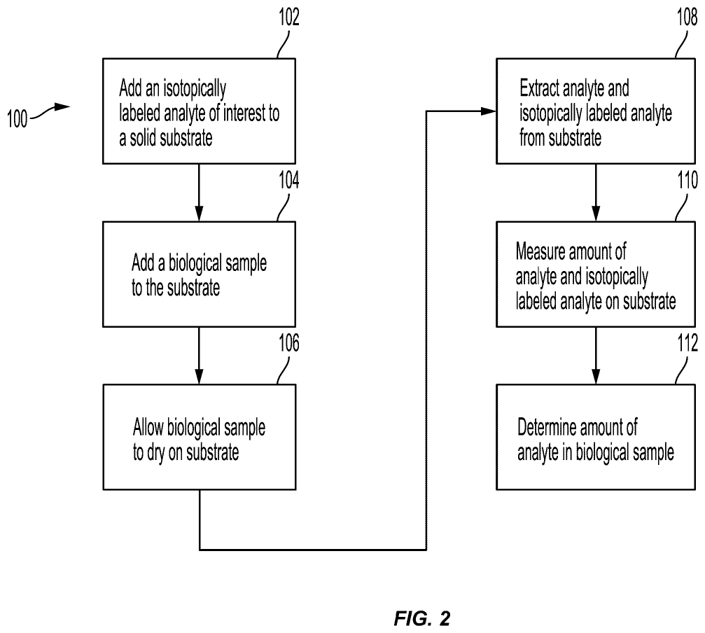

FIG. 2 shows a method of quantifying an analyte using an isotopically labeled analyte according to an embodiment of the invention. As shown in FIG. 2, the method 100 may comprise the step 102 of adding an isotopically labeled version of the analyte of interest that corresponds to the analyte of interest to a solid substrate. For example, if the analyte of interest is testosterone, the isotopically labeled version of the analyte of interest can be an isotope of testosterone (e.g., ¹³C-testosterone). The analyte of interest may be any type of biomolecule and/or therapeutic agent that is suitable for detection with the methods employed. For example, the analyte of interest may comprise a protein, a peptide, a hormone or other small biomolecules, a therapeutic drug, a vitamin, a nucleic acid or any biomolecule of interest. In some embodiments, the analyte of interest can be any biomolecule in a biological sample (e.g., whole blood).

Next, the method 100 may comprise the step 104 of adding a biological sample from a subject to the solid substrate. The sample may be self-collected by the subject. Examples of suitable biological samples include, but are not limited to, blood or a blood product (e.g., serum, plasma, or the like), urine, nasal swabs, saliva, or a liquid biopsy sample. In some embodiments, the biological sample can be collected using a microsampling device.

Next, the biological sample may be allowed to dry on the substrate 106. In an embodiment, the biological sample may be separated into different components on the substrate prior to drying. For example, blood may be deposited onto a sampling paper substrate which limits migration of red blood cells allowing for separation of the blood and plasma fractions prior to drying in order to produce a dried plasma sample for analysis. Once the biological sample has dried on the substrate, the subject may send the substrate with the applied sample to a laboratory for testing.

The method 100 may include exposing the solid substrate to an extraction solution to extract the analyte of interest and the isotopically labeled version of the analyte of interest from the solid substrate 108. As the analyte of interest and the isotopically labeled version of the analyte of interest are the same compound (i.e., with minor modifications to the number of neutrons within certain elements in the isotopically labeled analyte), they should behave identically during extraction. In this way, an extraction of less than 100% of the analyte of interest does not lead to bias in the assay. The extraction solution may be varied depending on the analyte of interest. Suitable extraction solutions may include water, buffers, alcohols, non-aqueous solvents and/or mixed aqueous-non-aqueous solutions. In certain embodiments, a protein solution (e.g., BSA or other carriers) may be added. Or in other embodiments, a protease may be added. In some embodiments, the extraction solution may include the isotopically labeled version of the analyte of interest. For example, a portion of the isotopically labeled version of the analyte of interest can be provided in the extraction solution. In some embodiments, the extraction solution may include a second isotopically labeled version of the analyte of interest that is different from the first isotopically labeled version of the analyte of interest disposed on the solid substrate. In this way, the amount of isotopically labeled version of the analyte of interest bound to (or absorbed by) the substrate during extraction may be quantified.

The method 100 may include measuring the amount of the analyte of interest and the isotopically labeled version of the analyte of interest 110 extracted from the solid substrate. In an embodiment, the method of measurement may comprise mass spectrometry (MS), or tandem mass spectrometry (MS/MS), or liquid chromatography-tandem mass spectrometry (LC-MS/MS). Or other analytical methods may be used.

The method 100 may include determining the amount of the analyte of interest present in the biological sample based on the amounts of the analyte of interest and the isotopically labeled version of the analyte of interest 112. The determination may be made in part as disclosed in detail herein.

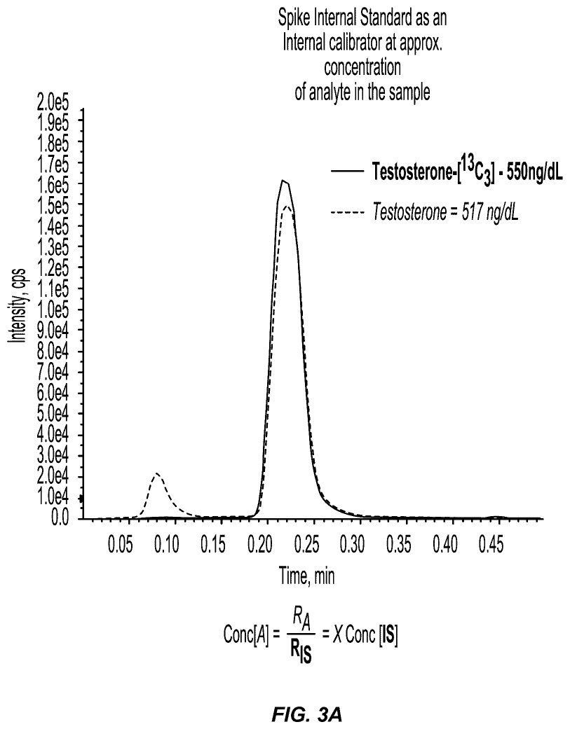

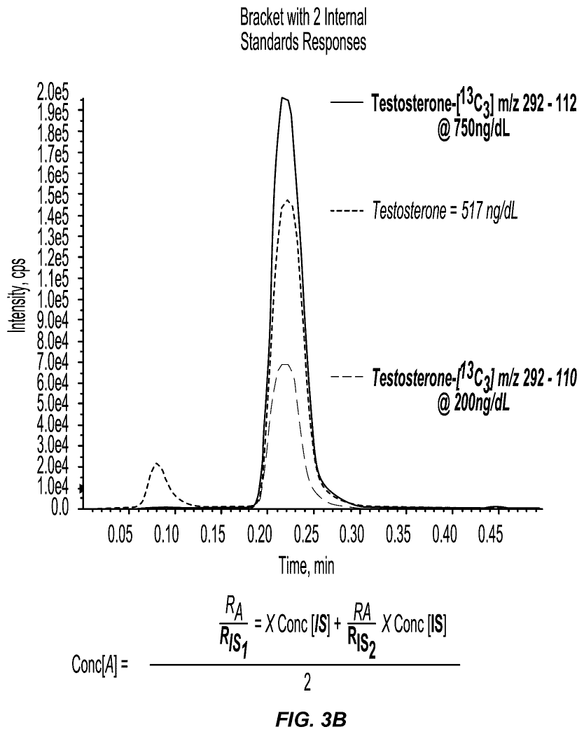

The isotopically labeled version of the analyte of interest may be added to the solid substrate at any concentration and/or concentrations. In an embodiment, the isotopically labeled version of the analyte of interest may be added to the solid substrate at a concentration that is near the anticipated concentration of the analyte of interest. For example. FIG. 3A shows a graph of peak responses for an analyte of interest (testosterone) and an isotopically labeled version of the analyte of interest (testosterone-[¹³C₃]) added to a solid substrate for internal calibration. In this embodiment, a known concentration of the isotopically labeled version of the analyte of interest (testosterone-[¹³C₃]) is mixed with an unknown sample, and the resulting measured peak areas or responses can be used to calculate the concentration of the analyte. The amount of the analyte of interest [A] may be determined as the measured level of A (R_{A}) divided by the measured level of the isotopically labeled version of the analyte of interest R_{IS} times the concentration of isotopically labeled version of the analyte of interest [IS] added to the substrate as shown in Equation (1).

$$Conc [ A ] = \frac{{R}_{A}}{{R}_{IS}} \times Conc [ IS ] \qquad Equation ( 1 )$$

The ratio of analyte to the isotopically labeled version of the analyte are determined using mass spectrometry, similar to the y-axis measurement performed for external calibration with internal standardization shown in FIG. 1. However, this mode of internal calibration does not include a set of external calibrators. The amount or concentration of the isotopically labeled version of the analyte is known, serving as a reference point or internal calibrator. Thus, a ratio of measured responses or integrated peak areas multiplied by the amount of concentration of the isotopically labeled version of the analyte added to the substrate enables the concentration or amount of the analyte to be computed. The accuracy of the calculated concentration of the analyte is higher if the measured peak area or response of the isotopically labeled version of the analyte of interest is close to the peak area or response of the analyte of interest. However, this can require prior knowledge of the approximate concentration of the analyte of interest, usually initially determined using external calibration with internal standardization and thus requiring two or more separate measures of the analyte concentration using two separate techniques and two or more separate analysis.

FIG. 3B shows peak responses for an analyte of interest and an isotopically labeled version of the analyte of interest added to a solid substrate. The graph shows peak responses for the isotopically labeled version of the analyte of interest (testosterone-[¹³C₃]) added to a solid substrate with two separate MS/MS transitions that appear at different peak intensities and thus are at different equivalent concentrations relative to the analyte concentration. Therefore, a single isotopically labeled version of the analyte of interest can be used to generate two discrete peak areas or responses for internal calibration. In this embodiment, two product ions of an isotopically labeled version of testosterone (e.g., ¹³C₃ Testosterone) were produced with a mass to charge ratio (m/z) of 292-112 and 292-110, respectively. The selected product ions of the isotopically labeled version of the analyte (testosterone) differs in yield by almost four fold of the selected reaction monitoring transitions of mass to charge m/z 292-112 at an added known concentration of 750 ng/dl and a second transition at m/z. 292-110 at an added concentration of 200 ng/dL, based on peak integrated peak area or response determined (3½ fold lower) relative to the known amount added.

The same precursor ion can yield integrated peak areas or responses of different intensities by selecting alternate m/z transitions, e.g., (292-112 versus 292-110). The intensities can be assigned a concentration at two discrete levels, in this instance, above and below the response observed for the analyte integrated peak area or response using the transition m/z 289-109. In some embodiments, the two discrete responses of internal calibrator responses derived from two different transitions can be used to calculate the concentration of analyte in a measured sample relative to the response of the isotopically labeled version of the analyte of interest according to Equation 1 (as shown in FIG. 3A). In some embodiments, the two discrete responses of internal calibrator responses derived from two different transitions can be used to calculate the average of both responses using responses that bracket the measured response for the analyte (as shown in FIG. 3B) according to Equation 2 below. Specifically, the amount (e.g., concentration) of the analyte of interest [A] may be determined as the measured level of A (R_{A}) divided by the measured level of the isotopically labeled version of the analyte of interest R_{IS1} times the concentration of isotopically labeled version of the analyte of interest [IS1] (e.g., lower concentration) plus the measured level of A (R_{A}) divided by the measured level of the isotopically labeled version of the analyte of interest R_{IS2} times the concentration of isotopically labeled version of the analyte of interest [IS2] (e.g., higher concentration) added to the substrate and divided by 2, which is shown below:

$$Conc [ A ] = \frac{\frac{{R}_{A}}{{R}_{{IS}_{1}}} \times Conc [ IS 1 ] + \frac{{R}_{A}}{{R}_{{IS}_{2}}} \times Conc [ IS 2 ]}{2} \qquad Equation ( 2 )$$

In some embodiments, the isotopically labeled version of the analyte of interest may be added at two or more different concentrations to different regions of a substrate. The sample can be added to each region of substrate including the isotopically labeled version of the analyte of interest. For example, a known volume of sample can be deposited in the regions of the substrate including the isotopically labeled version of the analyte of interest. In this embodiment, a single isotopically labeled version of the analyte of interest may be used to generate separate responses that correspond to different concentrations. The concentrations of the isotopically labeled version of the analyte of interest may be chosen to bracket the expected concentration of the analyte. In some embodiments, a range of concentrations of the isotopically labeled version of the analyte of interest may be used, thereby generating a calibration curve on a single solid substrate. In this method for internal calibration of a dried sample, the isotopically labeled version of the analyte(s) of interest may be added at different concentrations, for example, as a low concentration (1) and a high concentration (2), where the measurement cutoff has a high and low range. The amount (e.g., concentration) of the analyte of interest [A] may be determined according to Equation 2.

In some embodiments, a plurality of isotopically labeled versions of the analytes of interest (e.g., each having different isotopes of differing mass) may be added to the solid substrate. For example, in an embodiment, the method may comprise adding a second isotopically labeled version of the analyte of interest to the solid substrate, wherein the second isotopically labeled version of the analyte of interest comprises a stable isotope that is different from the isotope of the first isotopically labeled version of the analyte of interest. In some embodiments, the concentration of the second isotopically labeled version of the analyte of interest added to the substrate can be different than the first isotopically labeled version of the analyte of interest. For example, two differentially labeled versions of the same analyte of interest (e.g., one analyte labeled with ²D and one analyte labeled with ¹³C or different combinations thereof) may be added to the substrate at different concentrations.

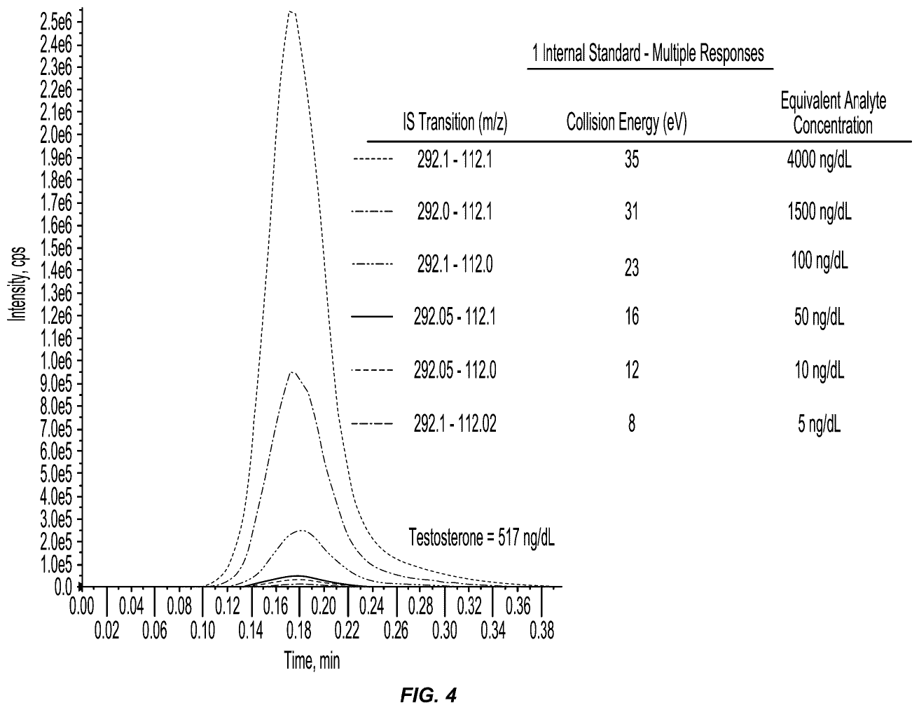

Alternatively, in some embodiments, the isotopically labeled version of the analyte of interest may be added at a single concentration and the measurement performed using an analytical technique (e.g., MS/MS) to provide a range of signal intensities for the isotopically labeled analyte that correspond to various analyte concentrations. As shown in FIG. 4, selecting for slight variations in either the precursor and/or product ion peaks of the isotopically labeled version of the analyte of interest and adjusting the collision energy in a mass spectrometer, the resulting signal intensity may mimic the presence of the analyte of interest over a range of concentrations. The peak area or responses of isotopically labeled version of the analyte of interest can be subsequently modified through changes in the collision energy or MS/MS transmission efficiency parameters for each transition, resulting in a larger number of peak area responses covering a wider “apparent” concentration range. In this embodiment, multiple peak areas or responses are generated from a single isotopically labeled version of the analyte of interest by generating near identical mass transitions. For example, as shown in FIG. 4, a mass spectrometer can fragment a precursor ion having a m/z of 292.1±0.1 amu to generate a product ion having a m/z of 112.1±0.1 amu. In some embodiments, any mass offset for a mass transition of an isotopically labeled version of the analyte of interest can be used to generate different peak areas or responses for the same isotopically labeled version of the analyte of interest. For example, the smallest mass difference for the isotopically labeled version of the analyte of interest can be used to generate a discrete transition for measurement, which may depend on software system of the mass spectrometer. In some embodiments, the precursor ion and/or precursor ion of the isotopically labeled version of the analyte of interest can have a m/z that differs by a range from 0.0001 to 0.1, or any range in between. In some embodiments, the precursor ion and/or precursor ion of the isotopically labeled version of the analyte of interest can have a m/z that differs by 0.01, 0.02, 0.03, 0.04, 0.05, 0.06, 0.07, 0.08, 0.09, or 0.1. The approximate concentrations for each response can be determined through comparison to responses determined through analysis using known amounts or concentrations of analyte.

Each of the transitions of the isotopically labeled version of the analyte of interest shown in FIG. 4 can be value assigned against a calibration curve containing increasing concentrations of analyte. For example, Table 1 below provides the equivalent analyte concentration for each transition of the isotopically labeled internal calibrator (ILIC).

TABLE 1

| Analyte | Analyte Peak | IS Peak Area | |
|---|---|---|---|
| Concentration [A] | Area (Ra) | (Ris) | ILIC #1 |
| 2.5 | 7054.517 | 10532.26 | 3.732 |
| 5 | 11290.82 | 8825.85 | 3.908 |
| 10 | 23821.803 | 11314.437 | 4.750 |
| 50 | 95506.79 | 9710.595 | 5.084 |
| 200 | 456837.937 | 10603.594 | 4.642 |
| 1000 | 1970307.633 | 9122.446 | 4.630 |
| 2500 | 5253780.503 | 9596.876 | 4.567 |
| 5000 | 10390778.7 | 10347.679 | 4.979 |

| | |
|---|---|
| Mean IS Response Equivalent Conc (ng/dL) | 4.537 |
| Standard Deviation (% CV) | 10.548 |

In this example, eight (8) individual extractions were performed with increasing analyte concentration for each extract, prior to mass spectrometric measurement. The concentration of the isotopically labeled internal calibrator response [ILIC] is calculated for each of the 8 unlabeled analyte calibrator samples compared to isotopically labeled internal calibrator response and a mean concentration value is produced for the internal calibrator peak response, with associated variance (% CV). The concentration of the ILIC is calculated using Equation 3.

$$Conc [ ILIC ] = \frac{( [ ConA ] {R}_{IS}}{{R}_{A}} \qquad Equation 3$$

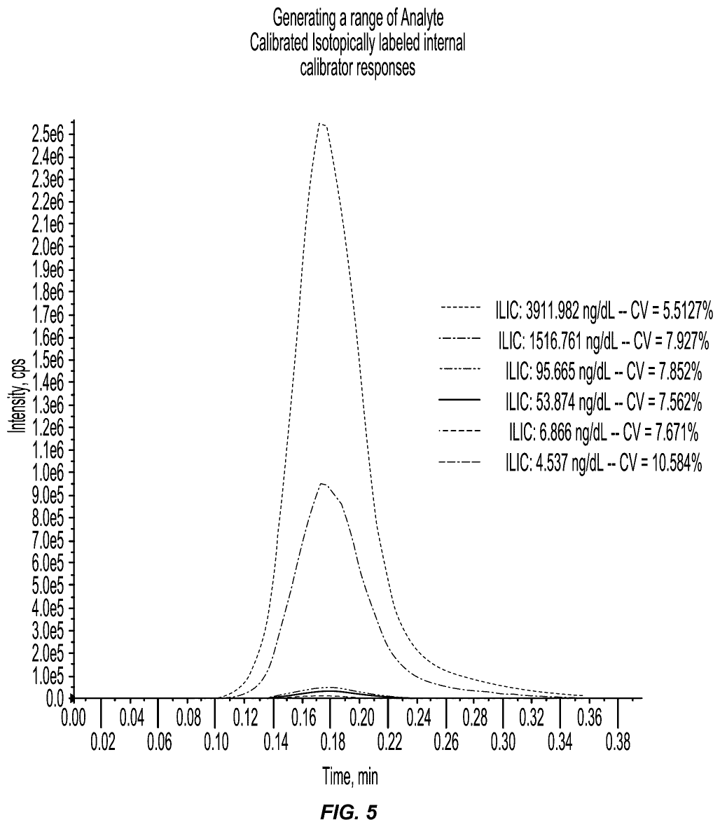

As shown in FIG. 5, each of the isotopically labeled internal calibrator peak areas or responses are individually calibrated against the analyte calibration curve process shown in Table 1 to generate an internal calibrator concentration for each response that spans a concentration range. The use of one or more analyte calibrators to assign isotopically labeled peak area or response concentrations is determined based on the apparent agreement across the measurement series determined. This process is further repeated to generate, via reverse calibration to the unlabeled analyte, internal calibrator concentrations for each response that spans a concentration range using the approaches described herein to generate different isotopically labelled internal calibrator responses.

In an embodiment, the signal intensities of the isotopically labeled internal calibrator may be chosen to correspond to medical decision points. For example, the medically relevant clinical cutoffs for the analysis of testosterone in serum and plasma based on age, gender, and pre or post-menopausal status, are shown in Table 2.

TABLE 2
Testosterone Medical Decision Points (MDP, ng/dL)

| Age (y) | Low | High |
|---|---|---|
| 1-10 | <3 | 10 |
| 11-18 (F) | <3 | 38 |
| 11-18 (M) | <3 | 970 |
| 20-50 (F Pre) | 10 | 55 |
| 20-50 (M) | 350 | 1030 |
| 60-80 (F Post) | 7 | 10 |
| 60-80 (M) | 7 | 40 |

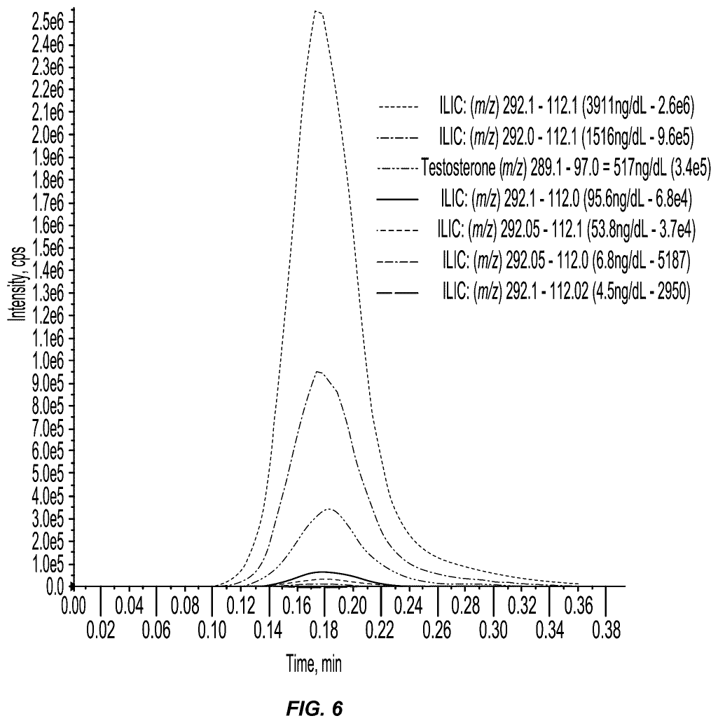

The isotopically labeled internal calibrator transitions in FIG. 6 cover the range of expected medically relevant concentration cutoffs for testosterone. Using the internal calibration series and the peak area measured for each internal calibration (ILIC 1-6) the concentration of unlabeled analyte (testosterone) can be computed, using either one or more internal calibration responses and assigned concentrations.

In yet other embodiments, the isotopically labeled analyte(s) of interest may be added at different concentrations to different substrates (e.g., cards). Thus, the method may comprise adding a different amount or amounts of the isotopically labeled analyte(s) of interest to a second solid substrate and performing the measurement of the analyte of interest with the second substrate and comparing amount of the analyte of interest determined with the first and second amounts of the isotopically labeled analyte(s) of interest.

A variety of methods may be used to measure the analyte of interest. In certain embodiments, the method may comprise liquid chromatography. Additionally, and/or alternatively, the method may comprise mass spectrometry. The mass spectrometry may comprise tandem mass spectrometry (MS/MS) and/or liquid chromatography tandem mass spectrometry (LC-MS/MS).

In certain embodiments, the use of tandem mass spectrometry may comprise generating multiple transitions for the isotopically labeled version of the analyte of interest and/or the analyte of interest. In certain embodiments, generating multiple transitions for the isotopically labeled version of the analyte of interest comprises generating a plurality different precursor and/or product ions for the isotopically labeled version of the analyte of interest.

The ability of MS/MS to generate multiple transitions for a single molecule allows for selection of transitions that closely bracket the analyte of interest. Measurements that are similar in size (i.e., mass units) and/or intensity (i.e., abundance) can increase the accuracy of measurement of any particular peak corresponding either to the analyte(s) of interest or the isotopically labeled analyte(s) of interest. In such embodiments, determining the amount of the analyte of interest present in the biological sample based on the amounts of the analyte of interest and the isotopically labeled version of the analyte of interest measured may comprise using multiple precursor and/or product ion peaks for the isotopically labeled version of the analyte of interest that have a measured response or a bracketing pair or a plurality of responses similar to the response generated for the precursor and/or product ion peak(s) for the analyte of interest. In this way, such multiple transitions may increase the power of the analysis by providing an inherent calibration curve for assessing the concentration of the analyte or analytes of interest.

Various methods may be used to generate the multiple transitions for the isotopically labeled version of the analyte of interest. In an embodiment, the method of generating multiple transitions for the isotopically labeled version of the analyte of interest may comprise generating alternate precursor ions by selecting for different ion source adducts or losses. For example, using a positive ion mode source, the analyte (M) may add a proton [M+H]+. Additionally, the analyte may also add (adduct) other ions to create e.g., [M+Na]+. [M+K]+, and/or [M+NH4]+, or other adducts. Or the analyte (M) may lose residues such as water (H₂O) to generate e.g., [M+H−H₂O]+. These different precursor ions result in different signal intensities and can be used to generate an inherent calibration system. In some embodiments, the transitions for the isotopically labeled version of the analyte of interest may comprise generating alternate precursor ions by using a negative ion source.

In certain embodiments, generating a plurality of different precursor and/or product ions may comprise measuring a plurality different isotopically labeled analytes wherein each of the different isotopically labeled analytes comprise a different isotope or different amounts of the same isotope. For example, the method may comprise the use of isotope walking, wherein precursor and/or product ions specific to known isotope variants are selected based in part on the presence of externally added isotopes and the contribution of naturally occurring isotopes. It is known that the contribution of certain isotopes is approximately as shown in Table 3. For example, it would be expected that a precursor ion peak of n corresponds to approximately 76% of the unlabeled naturally occurring analyte having a single chlorine-35 (³⁵Cl), with a precursor ion peak of n+2 corresponding to approximately 24% unlabeled analyte with chlorine-37 (³⁷Cl). Either chlorine isotope may be selected as a precursor ion or product ion to generate independent MS/MS responses for internal calibration using additionally isotopically labelled forms. In this case, a product ion for an analyte of interest that contains chlorine that is formed from an isotopically labeled internal standard (e.g., having multiple deuterium, nitrogen-15, oxygen-18 or carbon-13 atoms) is chosen that does not overlap the n and/or n+2 peaks due to ³⁵Cl and ³⁷Cl. In certain embodiments, the range of precursor and/or product peaks chosen by isotope walking may range from about 1-10 Daltons, or 1-6 Daltons.

TABLE 3

| Element | Atomic Mass | Relative Abundance (%) |
|---|---|---|
| H | 1 | 99.99 |
| | 2 | 0.01 |
| C | 12 | 98.93 |
| | 13 | 1.07 |
| N | 14 | 99.64 |
| | 15 | 0.36 |
| O | 16 | 99.76 |
| | 17 | 0.04 |
| | 18 | 0.20 |
| S | 32 | 94.99 |
| | 33 | 0.75 |
| | 34 | 4.25 |
| | 35 | 0.01 |
| Cl | 35 | 75.76 |
| | 37 | 24.24 |
| Br | 79 | 51.00 |
| | 81 | 49.00 |

Yet other methods may be used to generate multiple transitions for the isotopically labeled version of the analyte of interest and/or the analyte of interest. For example, generating multiple transitions for the isotopically labeled version of the analyte of interest and/or the analyte of interest may comprise use of minor mass offsets for precursor and/or product ions pairs. Such minor mass units may comprise selecting for mass units that differ by any amount of mass units (i.e., Daltons). An example is shown in FIG. 4. In certain embodiments, such minor offset transitions may be used in combination with applying modified collision energy settings to generate product ions of differing intensity for the minor mass offsets for the isotopically labeled analyte products such that the isotopically labeled version of the analyte of interest product ions of differing intensity may be calibrated to different concentrations of the analyte of interest. Thus, as shown in FIG. 4 by selecting for slight variations in either the precursor and/or product peaks and then adjusting the collision energy in a mass spectrometer, the resulting signal intensity may mimic the presence of the unlabeled analyte over a range of concentrations. Or the use of applying modified collision energy settings to generate product ions of differing intensity may be used independently of other techniques (e.g., isotope walking, the use of minor mass offsets). In some embodiments, use of applying modified collision energy settings to generate product ions of differing intensity may be used in conjunction with other techniques (e.g., isotope walking, the use of minor mass offsets). In certain embodiments, such precursor and/or product ion modifications resulting in a plurality of observed instrument responses (i.e. signals of differing intensity) for the isotopically labeled version of the analyte of interest may be calibrated to different concentrations of the analyte of interest to create a calibration scheme for an isotopically labeled analyte(s) to be used for concentration determination of the unlabeled analyte. In certain embodiments, the calibration curve may be designed to be informative with respect to medical decision points (FIG. 6).

To determine the amount of the analyte of interest in the biological sample, certain calibration approaches may be used. Thus, in certain embodiments, the method may comprise applying one or more calibration functions for determining the amount of the analyte of interest in the biological sample. For example, where multiple calibration values are used, the multiple calibration values may be determined using a PADÉ calibration function (Pagliano et al., Calibration graphs in isotope dilution mass spectrometry; Analytica Chimica Acta, 896:63-67 (2015 Oct. 8) available on-line at https://doi.org/10.1016/j.aca.2015.09.020). Or other methods such as linear fit with calibration extrapolation, quadratic fit without calibration extrapolation, concentration determination using direct comparison to the closest response or averaging of the concentration derived from the closest pair of isotopically labeled internal calibrator responses, or comparison to multiple isotopically labelled internal calibrator responses to the unlabeled analyte response may be used.

### Systems

Also disclosed are systems to measure the amount of an analyte of interest in a dried sample.

In certain embodiments, the system may comprise a device for measuring the amount of an analyte of interest present in a biological sample comprising at least one solid substrate, wherein a known amount of a first isotopically labeled version of the analyte of interest is applied to the solid substrate, and wherein the isotopically labeled version of the analyte of interest is the analyte of interest labeled with at least one stable isotope. In certain embodiments, the device (i.e., system) may comprise more than one isotopically labeled version of the analyte of interest applied to the solid substrate. Thus, in certain embodiments, the device may comprise an additional isotopically labeled analyte added to the solid substrate, wherein the additional isotopically labeled analyte comprises the analyte of interest labeled with an additional isotope that is the same as the at least one isotope and/or a different stable isotope. For example, a second isotopically labeled analyte may be added to the solid substrate, wherein the second isotopically labeled analyte comprises the analyte of interest labeled with a second or a different number of stable isotopes. In certain embodiments, the additional isotopically labeled version of the analyte of interest is present at a different concentration than the first isotopically labeled version of the analyte of interest. In such embodiments, the different isotopically labeled version of the analyte may be added to different regions (i.e., locations) on the solid substrate.

In certain embodiments, the system comprises a device for measuring the amount of a plurality of different analytes of interest present in a biological sample. For example, the system may comprise a known amount of a plurality isotopically labeled analytes of interest added to a solid substrate, wherein each of the plurality of isotopically labeled analytes of interest comprises one of the analytes of interest labeled with at least one stable isotope.

In various embodiments, multiple isotopes (e.g., 2, 3, 4, 5 or more) may be used. The multiple isotopes may be the same isotope (e.g., multiple deuterium or ²D atoms) or different isotopes (e.g., ²D and ¹³C).

In certain embodiments, the isotopically labeled analyte(s) of interest may be added to solid substrate at a concentration or concentrations that are close to the expected concentration of the analyte of interest and/or are at or near to a medical decision point. For example, a first and second isotopically labeled analyte may added to the solid substrate at concentrations that fall on either side of a medical decision point for the unlabeled analyte. Or in some embodiments, several (i.e., more than two) different isotopically labeled analytes of interest, each having different isotopes of differing mass, and present at a plurality of different concentrations, may be added to the solid substrate. In this way, a calibration curve, comprising the isotopically labeled analytes of interest each labeled with a different isotope and present at various concentrations, may be included on a single substrate. Additionally. and/or alternatively, the system may comprise a plurality of solid substrates, or a single substrate with a plurality of different regions, each comprising a different amount of the one or more isotopically labeled version of the analyte of interest. In certain embodiments, the solution used to extract the analyte and isotopically labeled version of the analyte of interest may include a second differentially labelled internal standard.

In yet other embodiments, the system may comprise a station and/or component for performing any of the methods or steps of the methods disclosed herein and/or using any of the devices as disclosed herein. Thus, in an embodiment, the system may comprise: a station and/or component for adding a known amount of an isotopically labeled analyte(s) of interest to a solid substrate, wherein the isotopically labeled analyte or analytes of interest is the analyte of interest labeled with one or more stable isotopes; a station and/or component for receiving a substrate after a subject has added the biological sample to the substrate; a station and/or component for extracting the analyte or analytes of interest and the isotopically labeled analyte or analytes of interest from the substrate; a station and/or component for measuring an amount of the analyte or analytes of interest and the isotopically labeled analyte or analytes of interest present on the substrate; and/or a station and/or component determining the amount of the analyte or analytes of interest present in the biological sample based on the amounts of the analyte or analytes of interest and the isotopically labeled analyte or analytes of interest.

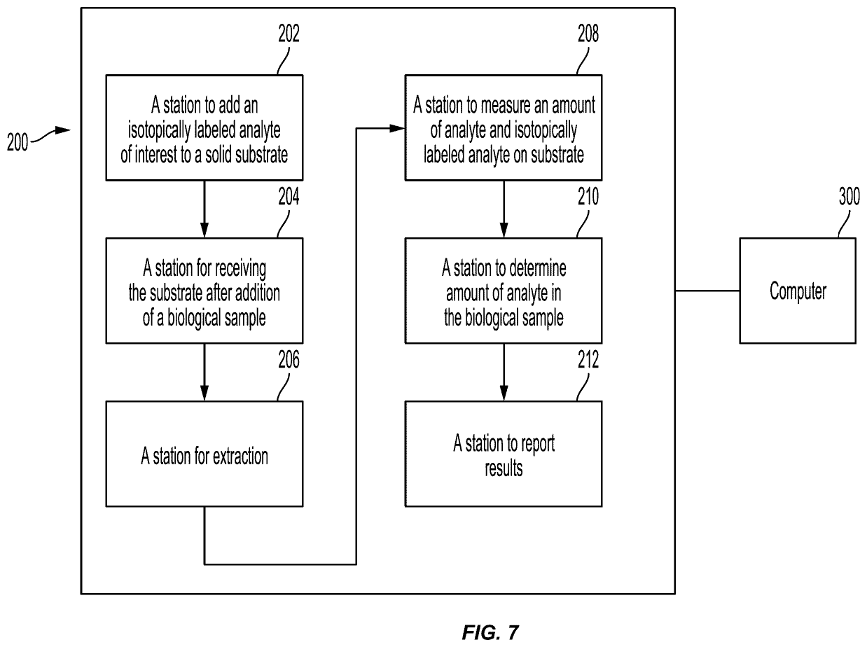

FIG. 7 shows a system 200 of the invention. The system may comprise a station and/or component 202 to add an isotopically labeled version of the analyte of interest to a solid substrate, wherein the isotopically labeled version of the analyte of interest is the analyte of interest labeled with at least one stable isotope. The analyte or analytes of interest may be any type of biomolecule or therapeutic agent that is suitable for detection with the methods employed. For example, the analyte(s) of interest may comprise a protein, a peptide, a hormone or other small biomolecules, a therapeutic drug, a therapeutic drug metabolite, a vitamin, a nucleic acid or any biomolecule of interest.

The system 200 may further comprise a station and/or component 204 for receiving a substrate after a subject has added the biological sample to the substrate. The sample may be self-collected by the subject and allowed to dry on the substrate. Examples of suitable biological samples include, but are not limited to, blood or a blood product (e.g., serum, plasma, or the like), urine, nasal swabs, saliva, or a liquid biopsy sample. In an embodiment, the sample body fluid (i.e., biological sample) may be allowed to separate into various components on the substrate prior to drying. For example, blood may be deposited onto a sampling paper substrate which limits migration of red blood cells allowing for separation of the blood plasma fraction prior to drying in order to produce a dried plasma sample for analysis. Once the sample has dried on the substrate, the subject may send the sample off to a laboratory for testing.

The system 200 may further comprise a station and/or component 206 for extracting the analyte or analytes of interest and the isotopically labeled analyte or analytes of interest from the substrate. As the analyte(s) of interest and the isotopically labeled analyte(s) of interest are the same compound (i.e., with minor modifications to the number of neutrons within certain elements in the isotopically labeled analyte), they should behave identically during extraction thereby reducing sample processing bias in the assay. The extraction solution may be varied depending on the analyte of interest. Suitable extraction solutions comprise water, buffers, alcohols, non-aqueous solvents and/or mixed aqueous-non-aqueous solutions. In certain embodiments, a protein solution (e.g., BSA or other carriers) may be added. Or in other embodiments, a protease may be added. In certain embodiments, the solution used to extract the analyte and internal calibrator may include a second differentially labelled internal standard.

The system 200 may further comprise a station and/or component 208 for measuring the amount of the analyte or analytes of interest and the isotopically labeled analyte or analytes of interest extracted from the substrate. In certain embodiments, the method of measurement may comprise mass spectrometry, tandem mass spectrometry (MS/MS), or liquid chromatography-tandem mass spectrometry (LC-MS/MS). Or other analytical methods may be used.

The system 200 may further comprise a station and/or component 210 determining the amount of the analyte of interest present in the biological sample based on the measured amounts of the analyte of interest and the isotopically labeled version of the analyte of interest. Thus, in certain embodiments, the system may comprise a component/station to quantify the measurement of an analyte of interest. Also, the system may comprise components/stations to perform statistical analysis of the data. In certain embodiments, the component/station may apply one or more calibration approaches for determining the amount of the analyte of interest in the biological sample. For example, where multiple calibration values are used, the multiple calibration values maybe determined using a PADE calibration function. Additionally, or alternatively, where multiple calibration values are used, the multiple calibration values may be determined using a linear fit, and/or a quadratic fit calibration function. Additionally, or alternately, analyte concentration may be generated through the determination of the ratio of unlabeled analyte to isotopically labelled analyte responses, multiplying by the known amount of isotopically labeled analytes added to the substrate. Additionally, or alternately, the analyte concentration may be generated through the determination of the mean of the ratio of unlabeled analyte to isotopically labeled analyte response for the two closest isotopically labeled responses multiplying by the effective amount of the two isotopically labeled analyte responses. Also, the system 200 may have a station and/or component 212 for reporting the results.

Any of the method steps and/or components and/or stations of the system may be controlled at least in part by a computer 300. Thus, also disclosed is a computer-program product tangibly embodied in a non-transitory machine-readable storage medium, including instructions configured to run any of the stations/components of the system and/or perform a step or steps of the methods of any of the disclosed embodiments. In one embodiment, the system comprises a computer-program product tangibly embodied in a non-transitory machine-readable storage medium, including instructions configured to identify the presence of and/or determine the amount of the analyte of interest in the extracted sample. Additionally, and/or alternatively, the computer program product may comprise instructions configured to identify the presence of and/or determine the amount of analyte of interest in the original sample. In some embodiments, the computer program product may comprise instructions for defining the measured analyte values.

Thus, in certain embodiments, the computer-program product may comprise instructions to quantify the measurement of an analyte of interest. For example, the computer-program product may comprise instructions to perform statistical analysis of the data. In certain embodiments, the instructions may comprise applying one or more calibration values for determining the amount of the analyte of interest in the biological sample. For example, where multiple calibration values are used, the multiple calibration values maybe determined using a PADE calibration function and/or a linear fit function and/or q quadratic fit calibration function. Additionally and/or alternatively, the system may instructions to quantify the measurement of an analyte of interest by the determination of the ratio of unlabeled analyte to isotopically labelled analyte responses, multiplying by the known amount of isotopically labeled analytes added to the substrate; and/or the determination of the mean of the ratio of unlabeled analyte to isotopically labelled analyte response for the two closest isotopically labelled responses multiplying by the effective amount of the two isotopically labeled analyte responses. Such results can then be reported to the subject providing the dried sample or their health care provider.

In some embodiments, the system further comprises a computer and/or a data processor configured to run the computer-program product tangibly embodied in a non-transitory machine-readable storage medium and/or any of the stations of the system. Thus, in certain embodiments, the system may comprise one or more computers, and/or a computer product tangibly embodied in a non-transitory computer readable storage medium containing instructions which, when executed on the one or more data processors, cause the one or more data processors to perform actions for performing any of the steps of the methods or implementing the systems or portions of the systems (e.g., components and/or stations) of any of embodiments disclosed herein. One or more embodiments described herein can be implemented using programmatic modules, engines, or components. A programmatic module, engine, or component can include a program, a sub-routine, a portion of a program, or a software component or a hardware component capable of performing one or more stated tasks or functions. As used herein, a module or component can exist on a hardware component independently of other modules or components. Alternatively, a module or component can be a shared element or process of other modules, programs or machines. For example, the system may comprise a computer for determining the measured analyte values. Thus, in certain embodiments, the system may comprise components to quantify the measurement of an analyte of interest. Also, the system may comprise components to perform statistical analysis of the data. In certain embodiments, the instructions may comprise applying one or more calibration values for determining the amount of the analyte of interest in the biological sample. For example, calibration values can be determined using a PADÉ calibration function; linear fit with calibration extrapolation: quadratic fit without calibration extrapolation, concentration determination using direct comparison to the closest response or averaging of the concentration derived from the closest pair of isotopically labeled analyte responses; or comparison to multiple isotopically labelled responses to the unlabeled analyte response may be used. In some embodiments, other calibration functions may be used. The computer and/or a data processor may then report results to the subject providing the dried sample or their health care provider.

### EXAMPLES

### Example 1—Internal Calibration Using the Highest and Lowest Amounts of Isotopically Labeled Versions of the Analyte of Interest

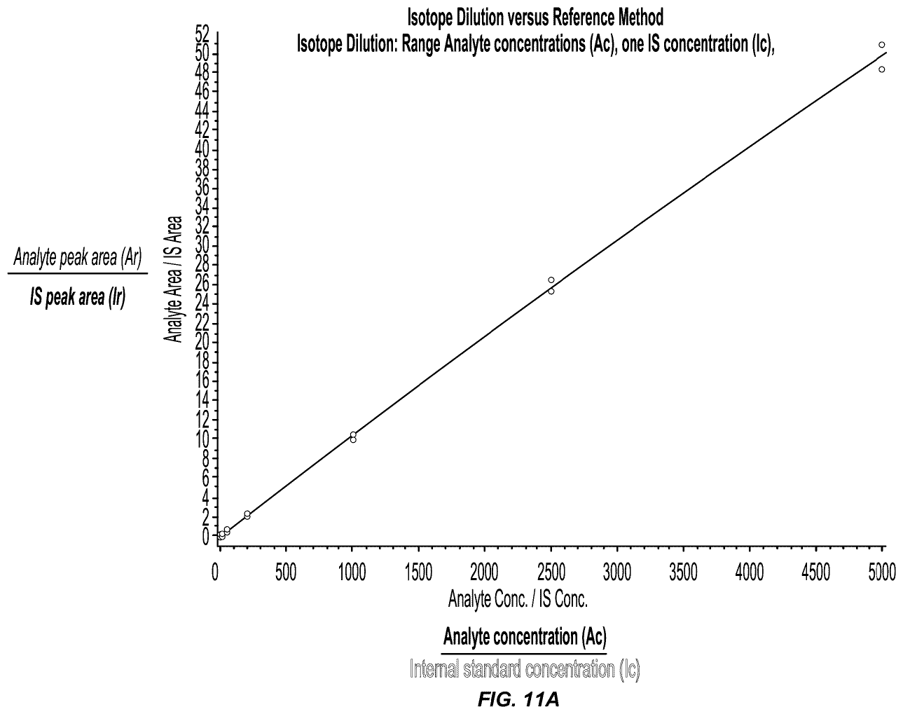

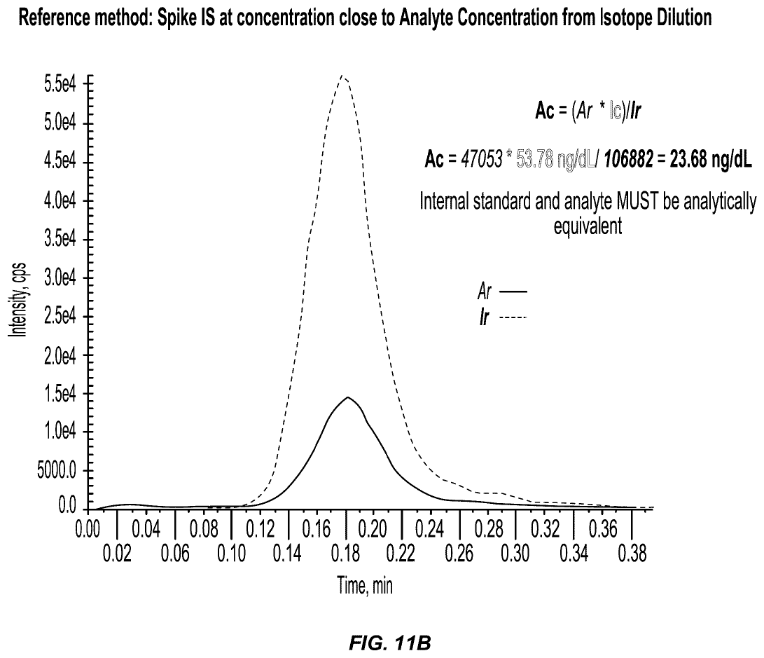

The methods for internal calibration for quantifying analytes in dried samples described herein were tested to determine accuracy of the methods. Specifically, the method of using one or more isotopically labeled versions of the analyte of interest on a solid substrate was tested for accuracy using Center of Disease Control (CDC) reference materials. The analyte concentrations were calculated using the internal calibration responses for the isotopically labeled versions of the analyte of interest according to Equation 4:

$$Ac = \frac{( Ar * Ic )}{Ir} , \qquad Equation 4$$

where Ac is the analyte concentration, Ar is the measured analyte peak area, Ic is the isotopically labelled internal calibrator concentration, and Ir is the measured isotopically labelled internal calibrator peak area. The reference method for calculating the analyte concentration is provided in FIGS. 11A and 11B. The internal calibration responses measured in FIG. 5 for isotopically labeled versions of testosterone were used to determine the accuracy of the method of internal calibration. Specifically, the lowest (4.537 ng/dL) and highest (3911.982 ng/dL) amounts of isotopically labeled internal calibrator responses from FIG. 5 were used to determine its accuracy for quantifying an analyte of interest.

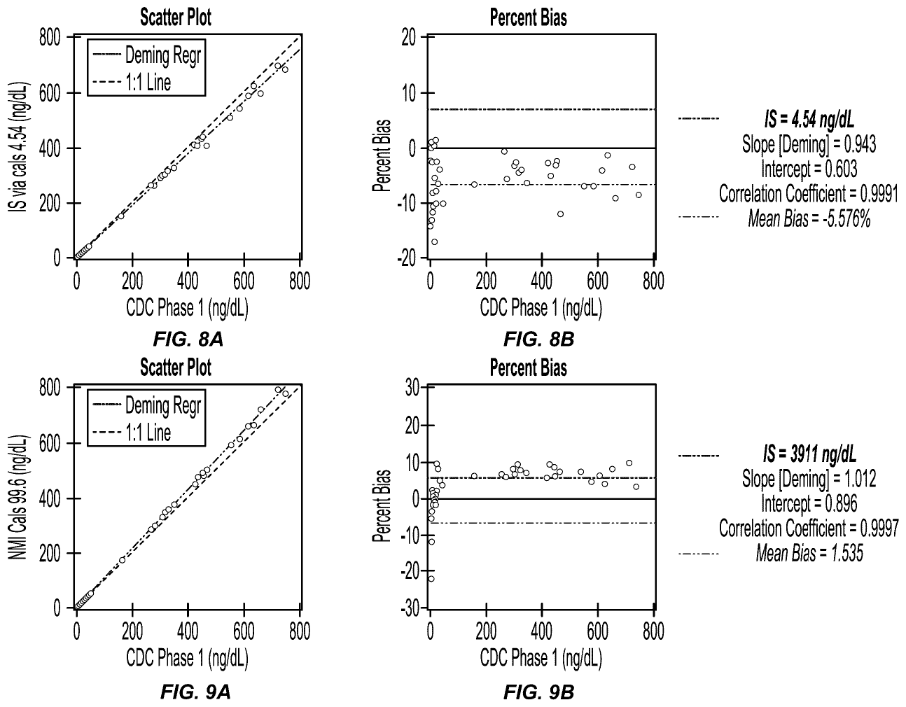

Serum samples were provided by the CDC with concentrations assigned using the CDC certified reference method procedure. FIG. 8A shows a scatter plot of the lowest amount of isotopically labelled internal calibrator response (ng/dL) (y-axis) versus the CDC results (ng/dL) (y-axis). Analysis of serum samples provided by the CDC with reference method value assignment (highest accuracy) and comparing the calculated concentrations observed using the lowest internal calibration response (transition, concentration 4.54 ng/dL) demonstrates a high correlation (r=0.9991, Slope [Deming]=0.943, Intercept=0.603 and mean bias=−5.576%). FIG. 8B shows a Bland Altman plot of lowest amount of isotopically labelled internal calibrator response (y-axis) versus the CDC reference standard. FIG. 8B shows that some specimens yielded individual negative biases >6.4%.

FIG. 9A shows a scatter plot of the highest amount of isotopically labelled internal calibrator response (ng/dL) (y-axis) versus the CDC reference standard (ng/dL) (y-axis). Using the highest amount of isotopically labelled internal calibrator response (concentration=3911 ng/dL) resulted in improved correlation (r=0.9997), Slope [Deming]=1.012, Intercept=0.896 and mean bias=1.535%. FIG. 9B shows a Bland Altman plot of the highest amount of isotopically labelled internal calibrator response (y-axis) from FIG. 5 versus the CDC reference standard (x-axis). FIG. 9B indicates some specimens yielded individual positive biases >6.4%.

### Example 2—Calibration Using the Mean Amount of Isotopically Labeled Versions of the Analyte of Interest

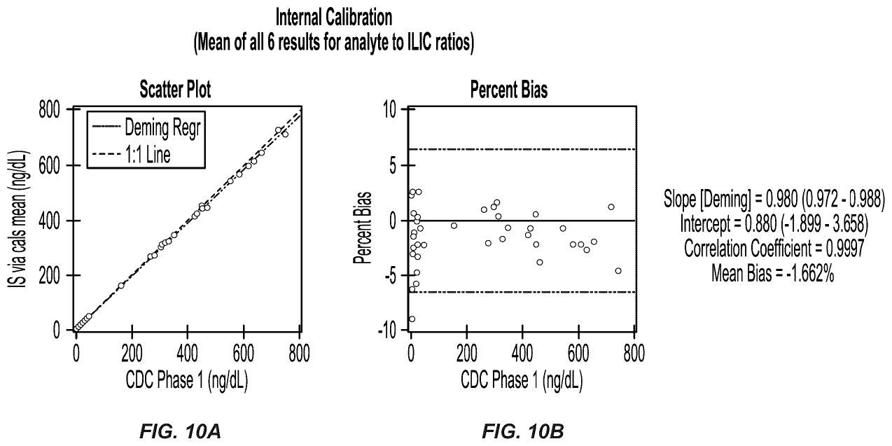

The methods for internal calibration for quantifying analytes in dried samples described herein using the mean value of the isotopically labeled versions of the analyte of interest were tested to determine accuracy of the method. The analyte concentrations were calculated using the mean value of the isotopically labeled versions of the analyte of interest according to Equation 4. The isotopically labelled internal calibrator response concentrations (Ic), and the measured isotopically labelled internal calibrator peak area (Ir) are taken from FIG. 5 above. The analyte concentration is calculated for each of the 6 in isotopically labelled internal calibrator response using Equation 4, and the mean value was determined. FIG. 10A shows a scatter plot of the mean value of the 6 isotopically labelled internal calibrator versions of the analyte of interest versus the CDC reference standard (ng/dL) (y-axis). Analysis of serum samples provided by the CDC with reference method value assignment (highest accuracy, x axis) and comparing the calculated concentrations observed using the mean concentration determined for the unlabeled analyte peak are responses against all 6 internal calibrator transitions previously calibrated demonstrated a high correlative agreement (r=0.9997), Slope [Deming]=0.980, Intercept=0.880 and mean bias=−1.662%. FIG. 10B shows a Bland Altman plot of mean of the isotopically labelled internal calibrator responses for each sample (y-axis) versus the CDC reference standard (x-axis). The Bland Altman plot indicates all samples demonstrated individual biases (10%) and 39 of 40 specimens yielded individual biases <6.4%.

If the isotopically labeled versions of the analyte of interest have large peaks or low peaks that vary substantially from the concentration of the analyte of interest, there can be higher error in the calculations above. Therefore, the average of the isotopically labeled versions of the analyte of interest with peaks closest to the peak of the analyte of interest can be used to calculate the analyte concentration. For example, the two closest peaks of the isotopically labeled versions of the analyte of interest can provide the least error for quantifying the analyte concentration. In some cases, the two closest peaks of the isotopically labeled versions of the analyte of interest that bracket the analyte concentration provide the most accurate analyte concentration. Additionally, and/or alternatively, the error may be less if the average of the two closest peaks of isotopically labeled versions of the analyte of interest are taken rather than the mean of all the peaks of the isotopically labeled versions of the analyte of interest.

### Illustrations

As used below; any reference to a series of illustrations is to be understood as a reference to each of those illustrations disjunctively (e.g., “Illustration 1-4” is to be understood as “Illustrations 1, 2, 3, or 4”).

Illustration 1 is a method of measuring an amount of an analyte of interest present in a dried biological sample comprising: a. adding a known amount of an isotopically labeled version of the analyte of interest to a solid substrate, wherein the isotopically labeled version of the analyte of interest comprises the analyte of interest labeled with at least one stable isotope: b. adding a biological sample to the substrate; c. allowing the biological sample to dry on the substrate; d. measuring an amount of the analyte of interest and the isotopically labeled version of the analyte of interest present on the substrate; and e. determining the amount of the analyte of interest present in the biological sample based on the measured amounts of the analyte of interest and the isotopically labeled version of the analyte of interest.

Illustration 2 is the method of any preceding or subsequent illustration, wherein the biological sample added to the substrate comprises a known volume.

Illustration 3 is the method of any preceding or subsequent illustration, wherein the analyte of interest and the isotopically labeled analyte are extracted from the solid substrate prior to measuring an amount of the analyte of interest and the isotopically labeled version of the analyte of interest present on the substrate.

Illustration 4 is the method of any preceding or subsequent illustration, wherein the isotopically labeled version of the analyte of interest is added to solid substrate at a concentration that is at or near an expected concentration of the analyte of interest or a medical decision point.

Illustration 5 is the method of any preceding or subsequent illustration, further comprising adding an additional isotopically labeled version of the analyte of interest to the solid substrate, wherein the additional isotopically labeled version of the analyte of interest comprises the analyte of interest labeled with an additional isotope that is the same as the at least one isotope and/or a different stable isotope.

Illustration 6 is the method of any preceding or subsequent illustration, wherein the additional isotopically labeled version of the analyte of interest is present at a different concentration than the first isotopically labeled version of the analyte of interest.

Illustration 7 is the method of any preceding or subsequent illustration, wherein measuring the amount of the analyte of interest and the isotopically labeled version of the analyte of interest present on the substrate comprises tandem mass spectrometry.

Illustration 8 is the method of any preceding or subsequent illustration, wherein the tandem mass spectrometry comprises generating multiple transitions for the isotopically labeled version of the analyte of interest.

Illustration 9 is the method of any preceding or subsequent illustration, wherein generating multiple transitions for the isotopically labeled version of the analyte of interest comprises generating a plurality different precursor and/or product ions for the isotopically labeled version of the analyte of interest.

Illustration 10 is the method of any preceding or subsequent illustration, wherein generating a plurality of different precursor and/or product ions comprises measuring a plurality different isotopically labeled analytes wherein each of the different isotopically labeled analytes comprise a different isotope or different amounts of the same isotope.

Illustration 11 is the method of any preceding or subsequent illustration, wherein the plurality of different precursor and/or product ions differ in mass in a range from 1 to 6 Daltons.

Illustration 12 is the method of any preceding or subsequent illustration, wherein generating a plurality of different precursor and/or product ions comprises selecting for different ion source adducts or ion source losses.

Illustration 13 is the method of any preceding or subsequent illustration, wherein generating a plurality of different precursor and/or product ions comprises use of minor mass offsets for precursor and/or product ions pairs.

Illustration 14 is the method of any preceding or subsequent illustration, wherein the plurality of different precursor and/or product ions differ in mass in a range from 0.0001 to 0.50 Daltons.

Illustration 15 is the method of any preceding or subsequent illustration, further comprising using different collision energies to generate pairs of precursor and product ions of differing intensity for the isotopically labeled version of the analyte of interest.

Illustration 16 is the method of any preceding or subsequent illustration, wherein the precursor and product ions pairs of differing intensity for the isotopically labeled version of the analyte of interest are calibrated to different concentrations of the analyte of interest.

Illustration 17 is the method of any preceding or subsequent illustration, further comprising applying one or more calibration values for determining the amount of the analyte of interest in the biological sample.

Illustration 18 is the method of any preceding or subsequent illustration, wherein the one or more calibration values are determined using a PADE calibration function, a linear calibration function and/or a quadratic calibration function.

Illustration 19 is a system for measuring an amount of an analyte of interest present in a biological sample comprising at least one solid substrate, wherein a known amount of a first isotopically labeled version of the analyte of interest is applied to the solid substrate, and wherein the first isotopically labeled version of the analyte of interest is the analyte of interest labeled with at least one stable isotope.

Illustration 20 is the system of any preceding or subsequent illustration, wherein the first isotopically labeled version of the analyte of interest is added to the solid substrate at a concentration that is at or near the expected concentration of the analyte of interest or a medical decision point.

Illustration 21 is the system of any preceding or subsequent illustration, further comprising a second isotopically labeled analyte added to the solid substrate, wherein the second isotopically labeled analyte comprises the analyte of interest labeled with an additional isotope that is the same as the at least one isotope and/or a different stable isotope.

Illustration 22 is the system of any preceding or subsequent illustration, wherein the second isotopically labeled analyte is present at a different concentration than the first isotopically labeled analyte.

Illustration 23 is the system of any preceding or subsequent illustration, wherein the first isotopically labeled version of the analyte of interest is disposed on a first region of the solid substrate and the second isotopically labeled version of the analyte of interest is disposed on a second region of the solid substrate.

Illustration 24 is the system of any preceding or subsequent illustration, wherein the first region of the solid substrate is separate and distinct from the second region of the solid substrate.

Illustration 25 is the system of any preceding or subsequent illustration, wherein the first isotopically labeled version of the analyte of interest is disposed on a plurality of regions on the solid substrate.

Illustration 26 is the system of any preceding or subsequent illustration, wherein the first isotopically labeled version is deposited on a first region of the solid substrate at a first concentration and a second isotopically labeled version is deposited on a second region of the solid substrate at a second concentration, wherein the first concentration is different from the second concentration.

That which is claimed is:

1. A method of measuring an amount of an analyte of interest present in a dried biological sample comprising:
a. adding a known amount of an isotopically labeled version of the analyte of interest to a solid substrate, wherein the isotopically labeled version of the analyte of interest comprises the analyte of interest labeled with at least one stable isotope;
b. adding a biological sample to the substrate;
c. allowing the biological sample to dry on the substrate;
d. measuring an amount of the analyte of interest and the isotopically labeled version of the analyte of interest present on the substrate; and
e. determining the amount of the analyte of interest present in the biological sample based on the measured amounts of the analyte of interest and the isotopically labeled version of the analyte of interest.

2. The method of claim 1, wherein the biological sample added to the substrate comprises a known volume.

3. The method of claim 1, wherein the analyte of interest and the isotopically labeled analyte are extracted from the solid substrate prior to measuring an amount of the analyte of interest and the isotopically labeled version of the analyte of interest present on the substrate.

4. The method of claim 1, wherein the isotopically labeled version of the analyte of interest is added to solid substrate at a concentration that is at or near an expected concentration of the analyte of interest or a medical decision point.

5. The method of claim 1, further comprising adding an additional isotopically labeled version of the analyte of interest to the solid substrate, wherein the additional isotopically labeled version of the analyte of interest comprises the analyte of interest labeled with an additional isotope that is the same as the at least one isotope and/or a different stable isotope.

6. The method of claim 5, wherein the additional isotopically labeled version of the analyte of interest is present at a different concentration than the isotopically labeled version of the analyte of interest.

7. The method of claim 1, wherein measuring the amount of the analyte of interest and the isotopically labeled version of the analyte of interest present on the substrate comprises tandem mass spectrometry.

8. The method of claim 7, wherein the tandem mass spectrometry comprises generating multiple transitions for the isotopically labeled version of the analyte of interest.

9. The method of claim 8, wherein generating multiple transitions for the isotopically labeled version of the analyte of interest comprises generating a plurality different precursor and/or product ions for the isotopically labeled version of the analyte of interest.

10. The method of claim 9, wherein generating a plurality of different precursor and/or product ions comprises measuring a plurality different isotopically labeled analytes wherein each of the different isotopically labeled analytes comprise a different isotope or different amounts of the same isotope.

11. The method of claim 10, wherein the plurality of different precursor and/or product ions differ in mass in a range from 1 to 6 Daltons.

12. The method of claim 9, wherein generating a plurality of different precursor and/or product ions comprises selecting for different ion source adducts or ion source losses.

13. The method of claim 9, wherein generating a plurality of different precursor and/or product ions comprises use of minor mass offsets for precursor and/or product ions pairs.

14. The method of claim 13, wherein the plurality of different precursor and/or product ions differ in mass in a range from 0.0001 to 0.50 Daltons.

15. The method of claim 9, further comprising using different collision energies to generate pairs of precursor and product ions of differing intensity for the isotopically labeled version of the analyte of interest.

16. The method of claim 15, wherein the precursor and product ions pairs of differing intensity for the isotopically labeled version of the analyte of interest are calibrated to different concentrations of the analyte of interest.

17. The method of claim 1, further comprising applying one or more calibration values for determining the amount of the analyte of interest in the biological sample.

18. The method of claim 17, wherein the one or more calibration values are determined using a PADÉ calibration function, a linear calibration function and/or a quadratic calibration function.

19. A system for measuring an amount of an analyte of interest present in a biological sample comprising:
at least one solid substrate; and
a known amount of a first isotopically labeled version of the analyte of interest disposed on the solid substrate, wherein the first isotopically labeled version of the analyte of interest is the analyte of interest labeled with at least one stable isotope.

20. The system of claim 19, wherein the first isotopically labeled version of the analyte of interest is added to the solid substrate at a concentration that is at or near an expected concentration of the analyte of interest or a medical decision point.

21. The system of claim 19, further comprising a second isotopically labeled analyte disposed on the solid substrate, wherein the second isotopically labeled analyte comprises the analyte of interest labeled with an additional isotope that is the same as the at least one isotope and/or a different stable isotope.

22. The system of claim 21, wherein the second isotopically labeled analyte is present at a different concentration than the first isotopically labeled version of the analyte of interest.

23. The system of claim 21, wherein the first isotopically labeled version of the analyte of interest is disposed on a first region of the solid substrate and the second isotopically labeled analyte is disposed on a second region of the solid substrate.

24. The system of claim 23, wherein the first region of the solid substrate is separate and distinct from the second region of the solid substrate.

25. The system of claim 19, wherein the first isotopically labeled version of the analyte of interest is disposed on a plurality of regions on the solid substrate.

26. The system of claim 19, wherein the first isotopically labeled version of the analyte of interest is deposited on a first region of the solid substrate at a first concentration and a second isotopically labeled version of the analyte of interest is deposited on a second region of the solid substrate at a second concentration, wherein the first concentration is different from the second concentration.
